(19) United States
(12) Patent Application Publication
(10) Pub. No.: US 2003/0088080 A1
(43) Pub. Date: May 8, 2003

(54) Receptor GPCRx10

(76) Inventors: Didier Communi, Vilvorde (BE); Vincent Lannoy, Brussels (BE); Cedric Govaerts, Brussels (BE); Marc Parmentier, Brussels (BE); Michel Detheux, Mons (BE)

Correspondence Address: PALMER & DODGE, LLP, KATHLEEN M. WILLIAMS / STR, 111 HUNTINGTON AVENUE, BOSTON, MA, 02199, US

(21) Appl. No.: 09/885,453

(22) Filed: Jun. 21, 2001

Related U.S. Application Data
(60) Provisional application No. 60/212,908, filed on Jun. 20, 2000.

(30) Foreign Application Priority Data
Dec. 5, 2000 (EP) EP 00 870 289.6

(51) Int. Cl. C07H 21/02; C07H 21/04; C07K 5/00; C07K 7/00; C07K 16/00; C07K 17/00; A61K 38/00

(52) U.S. Cl. 536/23.1; 530/324

(57) ABSTRACT

The present invention is related to a novel G-protein coupled receptor having an amino acid sequence which presents more than 75% sequence identity with the sequence SEQ ID NO. 1. The present invention further comprises a method for screening a substance as a potential agonist, reverse agonist, or antagonist to the receptor of the invention. The present invention further comprises a diagnostic method to identify expression of the receptor in target tissues or cells.

### FIELD OF THE INVENTION

The present invention is related to a newly identified member of the superfamily of G-protein-coupled receptors as well as to the various uses that can be made of the receptor.

The invention is also related to the polynucleic acid sequence (polynucleotide) encoding the receptor(s).

The invention is further related to methods using receptor polypeptide and polynucleotide applicable to diagnostic and treatment in receptor-mediated disorders.

The invention is further related to drug-screening methods using the receptor polypeptide and polynucleotide, to identify agonists and antagonists applicable to diagnostic, prevention and/or treatment of the various disorders.

The invention further encompasses unknown agonists and antagonists detected and recovered based on the receptor polypeptide and polynucleotide.

The invention is further related to procedures for producing the receptor polypeptide and polynucleotide according to the invention, preferably by genetic recombinant methods.

### BACKGROUND OF THE INVENTION

G-protein coupled receptors (GPCRs) are proteins responsible for transducing a signal within a cell. GPCRs have usually seven transmembrane domains. Upon binding of a ligand to an extra-cellular portion or fragment of a GPCR, a signal is transduced within the cell that results in a change in a biological or physiological property or behaviour of the cell. GPCRs, along with G-proteins and effectors (intracellular enzymes and channels modulated by G-proteins), are the components of a modular signalling system that connects the state of intra-cellular second messengers to extra-cellular inputs.

GPCR genes and gene products are potential causative agents of disease and these receptors seem to be of critical importance to both the central nervous system and peripheral physiological processes.

The GPCR protein superfamily is represented in five families: Family I, receptors typified by rhodopsin and the beta2-adrenergic receptor and currently represented by over 200 unique members; Family II, the parathyroid hormone/calcitonin/secretin receptor family; Family III, the metabotropic glutamate receptor family, Family IV, the CAMP receptor family, important in the chemotaxis and development of D. discoideum; and Family V, the fungal mating pheromone receptor such as STE2.

G proteins represent a family of heterotrimeric proteins composed of α, β and γ subunits, that bind guanine nucleotides. These proteins are usually linked to cell surface receptors (receptors containing seven transmembrane domains).

Following ligand binding to the GPCR, a conformational change is transmitted to the G protein, which caused the α-subunit to exchange a bound GDP molecule for a GTP molecule and to dissociate from the βγ-subunits.

The GTP-bound form of the α, β and γ-subunits typically functions as an effector-modulating moiety, leading to the production of second messengers, such as cAMP (e.g. by activation of adenyl cyclase), diacylglycerol or inositol phosphates.

Greater than 20 different types of α-subunits are known in humans. These subunits associate with a small pool of β and γ subunits. Examples of mammalian G proteins include Gi, Go, Gq, Gs and Gt. G proteins are described extensively in Lodish et al., Molecular Cell Biology,(Scientific American Books Inc., New York, N.Y., 1995), the contents of which are incorporated herein by reference.

Known and unknown GPCRs constitute now major targets for drug action and development.

Therefore, it exists a need for providing new G protein coupled receptors which could be used for the screening of new agonists and antagonists having advantageous potential prophylactic and therapeutical properties.

More than 300 GPCRs have been cloned thus far and it is generally assumed that it exists well over 1000 such receptors. Mechanistically, approximately 50-60% of all clinically relevant drugs act by modulating the functions of various GPCRs (Cudermann et al., J. Mol. Med., Vol. 73, pages 51-63, 1995).

### SUMMARY OF THE INVENTION

The present invention is related to newly identified member of G-protein-coupled receptor, preferably a human receptor, as well as to the polynucleotide sequence encoding the human receptor described hereafter (SEQ ID NO. 1 and 2).

The present invention is also related to other newly identified members of G-protein-coupled receptors, preferably human receptors, as well as to the polynucleotide sequence encoding the other human receptor described hereafter.

The present invention is also related to nucleotidic and/or amino acid sequence homologous to the sequences corresponding to the receptor described hereafter.

An homologous sequence (which may exist in other mammal species) means a sequence which presents a high sequence identity or homology (which presents an identity higher than 70%, 75%, 80%, 85%, 90% or 95%) with the complete human sequence described hereafter, and preferably characterised by a similar pharmacology. The percentage of sequence identity between the nucleic acid or amino acid sequences of the invention and related nucleic acid and/or amino acid sequences may be determined using techniques known to those of skill in the art. Many publicly available databases and computer programs exist for the comparison of sequence homology, and are thus useful in the present invention. Such programs include, but are not limited to BLAST (http://www.ncbi.nlm.nih.gov/BLAST/), LALIGN, FASTA, and CLUSTALW (http://workbench.sdsc.edu/).

Another aspect of the present invention is related to a specific active portion of the sequence. The active portion could be a receptor which comprises a partial deletion upon the complete nucleotide or amino acid sequence and which still maintains the active site(s) necessary for the binding of specific ligands able to interact with the receptor.

Homologous sequences of the sequence according to the invention may comprise similar receptors which exist in other animal (rat, mouse, dog, etc.) or specific human populations, but which are involved in the same biochemical pathway.

Such homologous sequences may comprise addition, deletion or substitution of one or more amino acids or nucleotides, which does not substantially alter the functional characteristics of the receptor according to the invention.

Thus, the invention encompasses also a receptor and corresponding nucleotide sequence having exactly the same amino acid or nucleotide sequences as shown in the enclosed sequence listing, as well as molecules which differ, but which are retaining the basic qualitative binding properties of the complete receptor according to the invention.

The invention is preferably related to the (human) receptor characterised by the complete nucleotide and amino acid sequences described hereafter, to unknown (and not previously described in the state of the art) agonist, reverse agonist and antagonist compounds or inhibitors of the receptor. Preferably, the inhibitors are antisens RNAs, rybozymes or antibodies (or specific hypervariable (FAB, FAB′2, . . . ) portions thereof) that bind specifically to the receptor or its encoding nucleotide sequence (i.e. that have at least a 10 fold greater affinity for the receptors than any other naturally occurring antibody). The specific antibodies are preferably obtained by a process involving the injection of a pharmaceutically acceptable preparation of such amino acid sequence into a animal capable of producing antibodies directed against the receptor.

For instance, a monoclonal antibody directed to the receptor according to the invention is obtained by injecting of an expression plasmid comprising the DNA encoding the receptor into a mouse and than fusing mouse spleen cells with myeloma cells.

The present invention is also related to the polynucleotide according to the invention, possibly linked to other expression sequences and incorporated into a vector (plasmids, viruses, liposomes, cationic vesicles, and the like) and host cells transformed by such vector.

The present invention is also related to the recombinant, preferably human receptor according to the invention, produced by such host cells according to the method well known by the person skilled in the art, as well as a functional assay (diagnostic kit) comprising all the means and media for the identification of the receptor, its nucleotide sequence, as well as agonist, reverse agonist, antagonist and inhibitor of the receptor or its nucleotide sequence. The diagnostic kit comprises preferably the following elements: the receptor, its encoding nucleotide sequence, antibodies directed against the receptor or its nucleotide sequence, as well as possible agonist, reverse agonist, antagonist or inhibitor compounds of the receptor. The diagnostic kit comprises means and media for performing the diagnostic preferably through a measure of dosage/activity of the receptor, by genetic analysis of the receptor nucleotide sequence, preferably by RT/PCR or by immuno-analysis, preferably by the use of antibodies directed against the receptor.

The present invention is also related to a transgenic non-human mammal comprising a partial or total deletion of the genetic sequence encoding the receptor according to the invention, preferably a non human mammal comprising an homologous recombination “knock-out” of the nucleotide sequence (polynucleotide) according to the invention or a transgenic non human mammal overexpressing above natural level the polynucleotide sequence.

The transgenic non-human mammal can be obtained by methods well known by the person skilled in the art, for instance by the one described in the document WO98/20112 using classical techniques based upon the transfection of embryonic stem cells, preferably according to the method described by Carmeliet et al., Nature, Vol. 380, p. 435-439, 1996.

Preferably, in the transgenic non human mammal overexpressing, the polynucleotide according to the invention or active portions thereof has been previously incorporated in a DNA construct with an inducible promoter allowing its overexpression and possibly with tissues and other specific regulatory elements.

The present invention provides screening assays to identify substances which modulate the activity of the receptor as described herein. As used herein, the “activity” of a receptor refers to the function of the receptor in mediating a cellular response to an extracellular signal. Non-limiting examples of functions that constitute activity include stimulation of GDP for GTP exchange in a G-protein, kinase activation, protease activation, phosphatase activation and stimulation of protein:protein interaction. In addition, the “activity” of a receptor is reflected in the signaling function or the activity of downstream signaling pathways, or ultimately, in changes in the expression of one or more genes. Wild-type receptor activity is regulated, in that the activity is modulated in response to a ligand or agonist. A receptor exhibits “constitutive” activity if it is active in the absence of a ligand or agonist. Constitutive activity need not be particularly high level, but must be greater than the activity level of the wild type receptor in the absence of ligand or agonist. In addition to the receptor of the present invention having the amino acid sequence set forth in SEQ ID NO: 1, the present invention also encompasses an active portion of the amino acid sequence of SEQ ID NO: 1 wherein the active portion is defined as having the same or equivalent “activity” as the whole receptor having the entire sequence of SEQ ID NO: 1 as defined herein.

Another aspect of the present invention is related to a method and kit for performing the method for the screening (detection and possibly recovering) of compounds or a natural extract which are unknown (not yet described in the state of the art) or not known to be agonists, reverse agonists, antagonists or inhibitors of natural compounds to the activity of the receptor according to the invention., the method comprising: (a) contacting a cell or cell extract from the cell transfected with a vector expressing the polynucleotide encoding the receptor according to the invention or active portion(s) thereof, possibly isolating a membrane fraction from the cell extract or the complete cell with a compound or molecules present in the natural extract under conditions permitting binding of the compound or the mixture of molecules to the receptor, possibly by the activation of a functional response; and (b) detecting the presence (and possibly the binding) of the compound or the mixture of molecules to the receptor by means of a bioassay, (preferably a modification in the production of a second messenger or an increase in the receptor activity) in the presence of another compound working as an agonist, reverse agonist, antagonist or inhibitor to the receptor according to the invention and thereby possibly recovering and determining whether the compound or mixture of molecules is (are) able to work as agonist, reverse agonist, antagonist, or inhibitor of the compound to its receptor. According to the present invention, a reduction of receptor activity in the presence of a candidate reverse agonist, antagonist or inhibitor of at least 10%, preferably 20%, 30%, 50%, 70% and up to 100% relative to the activity of the receptor in the absence of the candidate reverse agonist, antagonist, or inhibitor is indicative of the substance being an agonist, antagonist, or inhibitor of receptor activity. Conversly, an increase in receptor activity in response to a candidate agonist of at least 10%, preferably 20%, 30%, 50%, 70% and up to 100% relative to the activity of the receptor in the absence of the candidate agonist is indicative of the substance being an agonist of receptor activity.

Preferably, the second messenger assay comprises the measurement of intra-cellular cAMP, intracellular inositol phosphates, intra-cellular diacylglycerol concentrations, arachinoid acid concentration, MAP kinase(s) or tyrosine kinase(s) pathways activation or intra-cellular calcium mobilisation.

The screening method according to the invention could be performed by well known methods to the person skilled in the art, preferably by high-throughput screening, diagnostic and dosage devices based upon the method described in the International patent application WO00/02045 performed upon various solid supports such as micro-titer plates or biochips (microarrays) according to known techniques by the person skilled in the art.

The present invention is also related to the known or unknown compound or molecule(s) characterised and possibly recovered by the method for its (their) use as a medicament in therapy and is related to the pharmaceutical composition comprising a sufficient amount of the compound or molecule and a pharmaceutically acceptable carrier or diluent for the preparation of a medicament in the prevention and/or the treatment of various diseases.

In the pharmaceutical composition, the carrier or the adequate pharmaceutical carrier or diluant can be any solid, liquid or gaseous support which is non-toxic and adapted for the administration (in vivo or ex vivo) to the patient, including the human, through various administration roots such as oral administration, intravenous administration, intradermal administration, etc.

The pharmaceutical composition may comprise also various vesicles or adjuvants well known by the person skilled in the art, able to modulate the immune response of the patient. The percentage of active compound/molecule(s) pharmaceutical carrier can vary, the range being only limited by the tolerance and the efficiency of the active compounds to the patient. The ranges of administration are also limited by the frequency of administration and the possible side effects of the compound or molecule(s).

In a further aspect of the invention, compounds or molecules identified by the screening methods of the invention are intended to be useful in the treatment or diagnosis of one or more of the following diseases: viral or bacterial infection, disturbances of cell migration, diseases or perturbations of the immune system, including cancer, development of tumours and tumour metastasis, inflammatory and neo-plastic processes, bacterial and fungal infections, for wound and bone healing and dysfunction of regulatory growth functions, pains, diabetes, obesity, anorexia, bulimia, acute heart failure, hypotension, hypertension, urinary retention, osteoporosis, angina pectoris, myocardial infarction, restenosis, atherosclerosis, diseases characterised by excessive smooth muscle cell proliferation, aneurysms, wound healing, diseases characterised by loss of smooth muscle cells or reduced smooth muscle cell proliferation, stroke, ischemia, ulcers, allergies, benign prostatic hypertrophy, migraine, vomiting, psychotic and neurological disorders, including anxiety, schizophrenia, maniac depression, depression, delirium, dementia and severe mental retardation, degenerative diseases, neurodegenerative diseases such as Alzheimer's disease or Parkinson's disease, and dyskinasias, such as Huntington's disease or Gilles de la Tourett's syndrome and other related diseases.

This invention relates to the use of a human G protein-coupled receptor as a screening tool to identify agonists, reverse agonists or antagonists of the aequorin luminescence resulting from expression of these receptors.

### DESCRIPTION OF THE DRAWINGS

FIG. 1 shows the results of a Northern blot analysis of GPCR×10 expression in human and dog thyroid glands.

### DETAILED DESCRIPTION

The present invention is based, in part, on the discovery of a novel seven-transmembrane domain, G-protein coupled receptor (GPCR), the amino acid sequence of which is shown in SEQ ID NO: 1. The present invention further relates to the nucleic acid sequence encoding the GPCR of the invention.

Identification of GPCR×10

The present invention relates to a novel GPCR related to the purinergic receptor P2Y. Sequences of the GPCRs GPR8 (GenBank Accession No. XP009663), ChemR23 (GenBank Accession No.CAA75112), HM74 (GenBank Accession No.I69202), and GPR14 (GenBank Accession No.Q9UKP6) were used as querries to search for homologies in public high-thoughput genomic sequence databases (http://www.ncbi.nlm.nih.gov/). Using this strategy, a novel human sequence of GPCR was identified and was named GPCR×10, the amino acid and nucleotide sequences of which are shown in SEQ ID Nos 1 and 2, respectively.

Homologous Sequences

In one aspect the present invention provides G-protein coupled receptors having an amino acid sequence of more than 75% sequence identity with the sequence of SEQ ID NO: 1. The invention further provides a GPCR having an amino acid sequence of more than 80%, 85%, 90%, 95%, and up to 100% sequence identity with the amino acid sequence of SEQ ID NO: 1. One of skill in the art can easily determine the identity of a GPCR having the above recited sequence identity, based on the novel GPCR×10 amino acid sequence disclosed herein and using one or more of numerous publicly available protein databases and sequence alignment programs.

Many publicly available databases and computer programs exist for the comparison of sequence homology, and are thus useful in the present invention. Such programs include, but are not limited to BLAST (http://www.ncbi.nlm.nih.gov/BLAST/), LALIGN, FASTA, and CLUSTALW (http://workbench.sdsc.edu/).

Vectors and Host Cells

In one embodiment, the present invention provides both vector constructs comprising a nucleic acid sequence encoding the GPCR×10 of the present invention, and one or more host cells comprising such a vector.

A “vector” for purposes of the present invention may be any vector known to those of skill in the art such as a plasmid or viral vector, into which a sequence of the invention (i.e., SEQ ID NO: 2) has been inserted, in a forward or reverse orientation. The construct also will include regulatory sequences, including, for example, a promoter, operably linked to the sequence. Large numbers of suitable vectors and promoters are known to those of skill in the art, and are commercially available. The following vectors are provided by way of example. Bacterial: pQE70, pQE60, pQE-9 (Qiagen) pBs, phagescript, psiX174, pBluescript SK, pBsKS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, pXT1, pSG (Stratagene) pSVK3, pBPV, pMSG, pSVL (Pharmacia). However, any other plasmid or vector may be used as long as it is replicable and viable in the host.

Promoter regions can be selected from any characterized gene and incorporated into appropriate vectors using techniques well known in the art. Two appropriate vectors are pKK232-8 and pCM7. Particular named bacterial promoters include lac, lacZ, T3, T7, gpt, PR, PL and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

A host cell containing an above-described construct may be a higher eukaryotic cell, such as a mammalian cell, or a lower eukaryotic cell, such as a yeast cell, or the host cell may be a prokaryotic cell, such as a bacterial cell. Introduction of the construct into the host cell can be effected by calcium phosphate transfection, DEAE-Dextran mediated transfection, liposome mediated transfection, or electroporation (Ausubel et al., supra, 1992, pp. 9-5 to 9-14). The constructs in host cells can be used in a conventional manner to produce the gene product encoded by the recombinant sequence (i.e., GPCR×10). Alternatively, the polypeptides of the invention can be synthetically produced by conventional peptide synthesizers.

Polypeptides can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook et al., Molecular Cloning: A Laboratory Manual, Second Edition, 1989, (Cold Spring Harbor Press, Cold Spring Harbor, N.Y.), the disclosure of which is hereby incorporated by reference.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), alpha factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

Transcription of a DNA encoding the polypeptides of the present invention by higher eukaryotes is increased by inserting an enhancer sequence into the vector. Enhancers are cis-acting elements of DNA, usually about from 10 to 300 bp, that act on a promoter to increase its transcription. Examples include the SV40 enhancer on the late side of the replication origin (bp 100 to 270), a cytomegalovirus early promoter enhancer, a polyoma enhancer on the late side of the replication origin, and adenovirus enhancers. Useful expression vectors for bacterial use are constructed by inserting a structural DNA sequence encoding a desired protein together with suitable translation initiation and termination signals in operable reading phase with a functional promoter. The vector may include one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice. As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, PKK223-3 (Pharmacia Fine Chemicals, Uppsala, Sweden) and GEM1 (Promega, Madison, Wis.). These pBR322 “backbone” sections are combined with an appropriate promoter and the structural sequence to be expressed.

Following transformation of a suitable host strain and growth of the host strain to an appropriate cell density, the selected promoter is derepressed by appropriate means (e.g., temperature shift or chemical induction) and cells are cultured for an additional period. Cells are typically harvested by centrifugation, disrupted by physical or chemical means, and the resulting crude extract retained for further purification. Microbial cells employed in expression of proteins can be disrupted by any convenient method, including freeze-thaw cycling, sonication, mechanical disruption, or use of cell lysing agents. Such methods are well-known to those skilled in the art.

Various mammalian cell culture systems can also be employed to express recombinant protein. Examples of mammalian expression systems include the COS-7 lines of monkey kidney fibroblasts, described by Gluzman, 1981, Cell, 23:175, and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, a polyadenylation site, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites, may be used to provide the required nontranscribed genetic elements. The expressed recombinant protein encoded by the gene sequence comprising the polynucleotide of the invention may be isolated, if necessary, by means known to those skilled in the art.

Transgenic Animals

In one embodiment, the present invention comprises a transgenic animal, or knock-out non-human mammal comprising a partial or total deletion of the genetic sequence encoding the receptor according to the invention. In an alternate embodiment, the invention comprises a transgenic non-human mammal which overexpresses the nucleic acid sequence encoding the receptor of the present invention above the natural level of the nucleic acid sequence normally found in the non-human mammal (i.e., a non-tansgenic animal).

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals described herein, the transgene causes cells to express a recombinant form of one of the subject polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques.

A transgenic animal of the invention can be created by introducing nucleic acid molecules encoding the polypeptides of the invention into the male pronuclei of a fertilized oocyte, e.g., by microinjection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of a polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the nucleic acid molecule of the invention, e.g., the transgene in its genome and/or expression of the transgene mRNA in tissues or cells of the animals. A transgenic founder animal can then be used to breed additional animals carrying the transgene. Moreover, transgenic animals carrying a transgene encoding polypeptides of the invention can further be bred to other transgenic animals carrying other transgenes.

In addition, transgenic non-human mammals may also be generated which are deficient for the nucleic acid sequence encoding the receptor of the invention. Such an animal is obtained by genetic modification leading to the partial or total deletion in the wild-type sequence through the integration of a foreigner nucleic acid sequence. The genetically modified sequence is incorporated in a vector of electroporated and reintroduced in an embryonic stem cell (ES) for which cellular clones are selected prior to integration into, preferably, a Swiss pseudo-gravide morula embryo according to the technique described by Carmeliet et al. (1996, Nature, 380: 435).

Method for Screening for a Candidate Agonist, Reverse Agonist, or Antagonist

The present invention further provides a screening assay to permit one of skill in the art to identify an agonist, reverse agonist, or antagonist of the receptor of the invention. As used herein, the term “agonist” means a molecule or composition that binds to and increases the activity of a receptor. An agonist includes, but is not limited to the natural ligand for a receptor. When an agonist is not the actual ligand, it can increase receptor activity in the absence of the natural ligand.

As used herein, the term “reverse agonist” means a molecule or composition that decreases the activity of a receptor below the baseline activity that receptor has in the absence of ligand or agonist. Unlike an antagonist, an inverse agonist does not function by blocking the activation by an agonist. Rather, a reverse agonist, on its own, reduces the baseline activity of the receptor.

As used herein, an “antagonist” refers to a molecule which decreases the activity by blocking the activation of the receptor by an agonist or natural ligand. An “antagonist” preferably reduces the activity of the receptor by at least 10%, 20%, 30%, 50%, 70%, 90%, and up to 100% compared to the activity of the receptor in the absence of the agonist.

The functional effect of a substance which acts as an agonist, reverse agonist, or antagonist may be determined in any of a number of ways, depending on the normal signalling pathways used by that receptor. Assays for alterations in ligand binding may be used, for example, as may assays to detect changes in downstream signalling pathway activity. The assay selected will also depend upon the receptor function one wishes to alter (e.g., constitutive activation of downstream signaling, enhanced agonist binding affinity, enhanced agonist potency, etc.). Assays include, for example, ligand binding assays, GTPase activation/GTP binding assays, adenylate cyclase assays, cAMP assays, assays for PI breakdown and DAG production, assays for IP3 levels, assays for PLC—activity, PKC activation assays, membrane polarization assays (Patch clamp), tyrosine kinase assays, MAP kinase assays, and reporter gene-based assays. For each assay, a comparison of activity of receptors in the presence or absence of a candidate agonist, reverse agonist, or antagonist allows a determination of the actual effect of the mutation predicted according to the methods of the invention.

A. Ligand Binding Assays:

When a ligand is known, one may use receptors expressed on a cell or use isolated membranes to evaluate the binding characteristics of labeled (e.g., radiolabeled or fluorescently labeled) ligand by a receptor in the presence of an agonist, reverse agonist, or antagonist relative to binding of ligand by cells or membranes containing receptor in the absence of an agonist, reverse agonist, or antagonist. The relative kinetics of binding may be evaluated, for example, by monitoring the displacement of labeled ligand by known quantities of unlabeled ligand on isolated membranes.

B. GTPase/GTP Binding Assays:

For GPCRs, a measure of receptor activity is the binding of GTP by cell membranes containing receptors. In the method described by Traynor and Nahorski, 1995, Mol. Pharmacol. 47: 848-854, incorporated herein by reference, one is essentially measuring G-protein coupling to membranes. Membranes isolated (using methods known in the art) from cells which express the receptor are incubated in a buffer containing 20 mM HEPES, pH 7.4, 100 mM NaCl, and 10 mM MgCl2, 80 pM ³⁵S-GTP S and 3 M GDP. The assay mixture is incubated for 60 minutes at 30° C., after which unbound labeled GTP is removed by filtration onto GF/B filters. Bound labeled GTP is measured by liquid scintillation counting.

GTPase activity is measured by incubating the membranes containing the receptor with γ³²P-GTP. Active GTPase will release the label as inorganic phosohate, which is detected in the supernatant by scintillation counting.

C. Downstream Pathway Activation Assays:

1. Adenylate Cyclase Assay:

Assays for adenylate cyclase activity are described by Maenhault et al., 1990, Biochem. Biophys. Res. Comm. 173: 1169-1178. Briefly, membranes containing approximately 50 to 75 μg of protein are incubated with a reaction medium containing 65 mM sucrose, 5 mM phosphocreatine, 10 U/ml creatine kinase, 0.04% BSA, 50 mM Tris, pH 7.4, 5 mM MgCl2, 0.25 mM EDTA, 0.12 mM RO20-1724 (phosphodiesterase inhibitor), 0.1 mM ATP, 0.1 mM GTP, and between 1.5 to 2.5 μCi/sample of γ³²P-ATP. Following the addition of the membrane mixture, the assay is incubated at 31° C. for 1 hour.

2. cAMP Assay:

Intracellular or extracellular cAMP is measured using a cAMP radioimmunoassay (RIA) or cAMP binding protein according to methods widely known in the art. For example, Horton & Baxendale, 1995, Methods Mol. Biol. 41: 91-105, which is incorporated herein by reference, describes an RIA for cAMP.

A number of kits for the measurement of cAMP are commercially available, such as the High Efficiency Fluorescence Polarization-based homogeneous assay marketed by LJL Biosystems and NEN Life Science Products.

3. Phospholipid Breakdown, DAG Production and Inositol Triphosphate Levels:

Receptors that activate the breakdown of phospholipids may be monitored for changes due to mutations predicted using the methods of the invention by monitoring either phospholipid breakdown, DAG production or Inositol triphosphate (IP₃) levels. Methods of measuring each of these are described in Phospholipid Signaling Protocols, edited by Ian M. Bird. Totowa, N.J., Humana Press, 1998, which is incorporated herein by reference.

An assay suitable to be adapted for monitoring receptor activated PI hydrolysis is also described by Sevva et al., 1986, Biochem. Biophys. Res. Comm. 140:160-166 and Peralta et al.,1988, Nature 334:434-437. Briefly, the functional assay involves labeling of cells with ³H-myoinositol for at least 48 hours. Following incubation with labeled myoinositol, the cells are lysed and the suspension is extracted with 3 ml of CHCl₃/MeOH (1:1). After centrifugation (3200 rpm for 5 min), the upper aqueous phase is removed and diluted with 2 ml H₂O and centrifuged again. The supernatants are loaded on columns containing 1 ml Dowex 1×8 AG resin previously equilibrated with 5 mM myoinositol and washed with 9 ml of 5 mM myoinositol followed by 8 ml of 60 mM sodium formate, 5 mM sodium borate. All of the inositol phosphates (IP1, IP2, IP3) are eluted together with 6 ml of 0.1 M formic acid, 1M ammonium formate. 3 ml of the eluates are removed and counted with 20 ml scintillation fluid for analysis.

4. PKC Activation Assays:

Growth factor receptor tyrosine kinases tend to signal via a pathway involving activation of Protein Kinase C (PKC), which is a family of phospholipid- and calcium-activated protein kinases. The PKC activation ultimately results in the transcription of an array of proto-oncogene transcription factor-encoding genes, including c-fos, c-myc and c-jun, proteases, protease inhibitors, including collagenase type I and plasminogen activator inhibitor, and adhesion molecules, including intracellular adhesion molecule I (ICAM I). Assays designed to detect increases in gene products induced by PKC may be used to monitor PKC activation and thereby receptor activity. In addition, the activity of receptors that signal via PKC may be monitored through the use of reporter gene constructs driven by the control sequences of genes activated by PKC activation. This type of reporter gene-based assay is discussed in more detail below.

5. Membrane Polarization Assays for Measurement of Receptor Activity:

Electrophysiological measurements of receptor activity may be performed using standard patch-clamp techniques, as described by Hoo et al. (1994, Receptors and Channels 2: 327), and summarized as follows.

Electrophysiological recordings are performed in a standard extracellular solution composed of 140 mM NaCl, 5.4 mM KCl, 1.0 mM MgCl₂, 1.3 mM CaCl₂, 5.0 mM HEPES and glucose to an osmolarity of 300 mOsm and pH adjusted to 7.2 with 1 mM NaOH.

For ion permeability studies, two other recording solutions are used, including a low calcium solution (140 mM NaCl, 1.0 mM MgCl2, 5.0 mM HEPES (pH 7.2 with NaOH)), and a low sodium solution (110 mM CaCl2, 1.0 mM MgCl2, 5.0 mM HEPES (pH to 7.2 with Ca(OH)₂).

Electrodes are constructed from thin-walled borosilicate glass (WPI Instruments), pulled to a fine point (1-2 μm in width) and filled with an intracellular solution composed of 140 mM CsCl, 1.0 mM MgCl2, 10 mM EGTA, 10 mM HEPES with pH adjusted to 7.2 with Cs(OH)2 and an osmolarity of 300 mOsm.

Whole cell voltage clamp recordings are carried out using an Axopatch 1B amplifier (Axon Instruments) or its equivalent. Agonists and antagonists are rapidly perfused over the cells through a multibarrel array of square glass tubes, the position of which is adjusted using a piezomotor under computer control. With this system it is possible to rapidly exchange solutions flowing over the cell and thus carry out extensive studies of receptor pharmacology.

6. Kinase Assays:

Assays for the activity of other signal transduction pathways regulated by a given receptor protein are known in the art. For example, direct assays for tyrosine kinase activity using known synthetic or natural tyrosine kinase substrates and labeled phosphate are well known, as are similar assays for other types of kinases (e.g., Ser/Thr kinases).

7. Transcriptional Reporters for Downstream Pathway Activation:

The intracellular signal that is transduced is generally initiated by the specific interaction of an extracellular signal, e.g., a ligand or agonist, with a receptor or ion channel present on the cell surface. This interaction sets in motion a cascade of intracellular events, the ultimate consequence of which is a rapid and detectable change in the transcription or translation of a gene. A mutation predicted using the methods of the invention will preferably have the same effect on downstream signalling as binding of agonist by the wild-type receptor. As used herein “promoter” refers to the transcriptional control elements necessary for receptor-mediated regulation of gene expression, including not only the promoter, but also any enhancers or transcription-factor binding sites necessary for receptor-regulated expression. By selecting promoters that are responsive to the intracellular signals resulting from agonist binding or an activating mutation, and operatively linking the selected promoters to reporter genes whose transcription, translation or ultimate activity is readily detectable and measurable, the transcription based reporter assay provides a rapid indication of whether a specific receptor or ion channel is activated.

Reporter genes such as luciferase, CAT or β-galactosidase are well known in the art, as are assays for detection of their products.

Transcription-based reporter assays can be used to test functional ligand-receptor or ligand-ion channel interactions for categories of cell surface-localized receptors including, but not limited to ligand-gated ion channels and voltage-gated ion channels, G protein-coupled receptors and growth factor receptors. Examples of each group include:

a) ligand-gated ion channels: nicotinic acetylcholine receptors, GABA (gamma-aminobutyric acid) receptors, excitatory receptors (e.g., glutamate and aspartate), and the like;

b) voltage-gated ion channels: calcium channels, potassium channels, sodium channels, NMDA receptor (actually a ligand-gated, voltage-dependent ion channel) and the like;

c) G protein-coupled receptors: adrenergic receptors, muscarinic receptors and the like. d) Growth factor receptors (Both RTKs and non-RTKs): Nerve growth factor NGF, heparin binding growth factors and other growth factors.

Transcriptional control elements include, but are not limited to, promoters, enhancers, and repressor and activator binding sites. Suitable transcriptional regulatory elements may be derived from the transcriptional regulatory regions of genes whose expression is rapidly induced, generally within minutes, of contact between the cell surface protein and the effector protein that modulates the activity of the cell surface protein. Examples of such genes include, but are not limited to, the immediate early genes (see, Sheng et al. (1990) Neuron 4: 477-485), such as c-fos. Immediate early genes are genes that are rapidly induced upon binding of a ligand to a cell surface protein. The induction of immediate early gene transcription does not require the synthesis of new regulatory proteins. The transcriptional control elements that are preferred for use in the gene constructs include transcriptional control elements from immediate early genes, elements derived from other genes that exhibit some or all of the characteristics of the immediate early genes, or synthetic elements that are constructed such that genes in operative linkage therewith exhibit such characteristics. The characteristics of preferred genes from which the transcriptional control elements are derived include, but are not limited to, low or undetectable expression in quiescent cells, rapid induction at the transcriptional level within minutes of extracellular simulation, induction that is transient and independent of new protein synthesis, subsequent shut-off of transcription requires new protein synthesis, and mRNAs transcribed from these genes have a short half-life. It is not necessary for all of these properties to be present.

One gene that is responsive to a number of different stimuli is the c-fos proto-oncogene. The c-fos gene is activated in a protein-synthesis-independent manner by growth factors, hormones, differentiation-specific agents, stress, and other known inducers of cell surface proteins. The induction of c-fos expression is extremely rapid, often occurring within minutes of receptor stimulation. This characteristic makes the c-fos regulatory regions particularly attractive for use as a reporter of receptor activation.

It is known in the art which receptors activate c-fos expression. One may determine whether an orphan receptor or an uncharacterized receptor activates the c-fos or other regulatory sequences by co-transfecting the wild-type receptor, under control of a strong promoter such as the CMV promoter/enhancer, with a c-fos (or other) reporter construct and measuring expression of the reporter in comparison to cells transfected with reporter alone (or with reporter and an expression vector minus receptor sequences). Generally, the overexpression of a receptor, even in the absence of ligand, will result in some signal transduction by the receptor.

The c-fos regulatory elements include (see, Verma et al., 1987, Cell 51: 513-514): a TATA box that is required for transcription initiation; two upstream elements for basal transcription, and an enhancer, which includes an element with dyad symmetry and which is required for induction by TPA, serun, EGF, and PMA.

The 20 bp transcriptional enhancer element located between −317 and −298 bp upstream from the c-fos mRNA cap site, is essential for serum induction in serum starved NIH 3T3 cells. One of the two upstream elements is located at −63 to −57 and it resembles the consensus sequence for cAMP regulation.

The transcription factor CREB (cyclic AMP responsive element binding protein) is, as the name implies, responsive to levels of intracellular cAMP. Therefore, the activation of a receptor that signals via modulation of cAMP levels may be monitored by measuring either the binding of the transcription factor, or the expression of a reporter gene linked to a CREB-binding element (termed the CRE, or cAMP response element). The DNA sequence of the CRE is TGACGTCA. Reporter constructs responsive to CREB binding activity are described in U.S. Pat. No. 5,919,649.

A CREB-responsive reporter construct is transfected into cells in the presence or absence of a candidate agonist, reverse agonist, or antagonist, and the relative level of receptor activity is determined by the reporter activities. Alternatively, the binding of CREB to DNA may be monitored using, for example, the well known electrophoretic mobility shift assay.

Other promoters and transcriptional control elements, in addition to the c-fos elements and CREB-responsive constructs, include the vasoactive intestinal peptide (VIP) gene promoter (cAMP responsive; Fink et al., 1988, Proc. Natl. Acad. Sci. 85:6662-6666); the somatostatin gene promoter (cAMP responsive; Montminy et al., 1986, Proc. Natl. Acad. Sci. 8.3:6682-6686); the proenkephalin promoter (responsive to cAMP, nicotinic agonists, and phorbol esters; Comb et al., 1986, Nature 323:353-356); the phosphoenolpyruvate carboxy-kinase (PEPCK) gene promoter (cAMP responsive; Short et al., 1986, J. Biol. Chem. 261:9721-9726); the NGFI-A gene promoter (responsive to NGF, cAMP, and serum; Changelian et al., 1989, Proc. Natl. Acad. Sci. 86:377-381); and others that may be known to or prepared by those of skill in the art.

Diagnostic Assays

It is contemplated that the activity of the novel GPCR disclosed herein may be modulated by potential agonists, reverse agonists, and antagonists which may significantly affect the function of a cell expressing the receptor. Therefore it is advantageous to provide a diagnostic assay for the identification of cells which contain the nucleic acid encoding the receptor, or cells express either the receptor protein.

Protein Detection

In one embodiment, the present invention provides a diagnostic assay to determine whether the GPCR×10 receptor of the present invention is expressed in a given cell population. Protein expression in cells may be determined using any technique known to those of skill in the art including but not limited to Western Blot, ELISA, immunoprecipitation, immunohistochemistry, and the like.

For example, the GPCR of the present invention may be detected in a cell by immunohistochemistry using an antibody which specifically binds to the GPCR of the invention or an immunogenic portion thereof.

A) Generation of antibodies

Antibodies that bind to the protein products encoded by a polynucleotide comprising a sequence of the invention are useful for protein purification, for the diagnosis and treatment of various diseases and for drug screening and drug design methods useful for identifying and developing compounds to be used in the treatment of various diseases. The term “antibody” is meant to encompass constructions using the binding (variable) region of such an antibody, and other antibody modifications. Thus, an antibody useful in the invention may comprise a whole antibody, an antibody fragment, a polyfunctional antibody aggregate, or in general a substance comprising one or more specific binding sites from an antibody. The antibody fragment may be a fragment such as an Fv, Fab or F(ab′)₂ fragment or a derivative thereof, such as a single chain Fv fragment. The antibody or antibody fragment may be non-recombinant, recombinant or humanized. The antibody may be of an immunoglobulin isotype, e.g., IgG, IgM, and so forth. In addition, an aggregate, polymer, derivative and conjugate of an immunoglobulin or a fragment thereof can be used where appropriate. Neutralizing antibodies are especially useful according to the invention for diagnostics, therapeutics and methods of drug screening and drug design.

Although a protein product (or fragment or oligopeptide thereof) derived from a polynucleotide comprising a sequence of the invention that is useful for the production of antibodies does not require biological activity, it must be antigenic. Peptides used to induce specific antibodies may have an amino acid sequence consisting of at least five amino acids and more conveniently at least ten amino acids. It is advantageous for such peptides to be identical to a region of the natural protein and they may contain the entire amino acid sequence of a small, naturally occurring molecule. Short stretches of amino acids corresponding to the protein product of a candidate gene of the invention may be fused with amino acids from another protein such as keyhole limpet hemocyanin or GST, and antibody will be produced against the chimeric molecule. Procedures well known in the art can be used for the production of antibodies to the protein products derived from the polynucleotide comprising a sequence of the invention.

For the production of antibodies, various hosts including goats, rabbits, rats, mice, etc., may be immunized by injection with the protein products (or any portion, fragment, or oligonucleotide thereof which retains immunogenic properties) of the candidate genes of the invention. Depending on the host species, various adjuvants may be used to increase the immunological response. Such adjuvants include but are not limited to Freund's, mineral gels such as aluminum hydroxide, and surface active substances such as lysolecithin, pluronic polyols, polyanions, peptides, oil emulsions, keyhole limpet hemocyanin, and dinitrophenol. BCG (bacilli Calmette-Guerin) and Corynebacterium parvum are potentially useful human adjuvants.

To generate polyclonal antibodies, the antigen protein may be conjugated to a conventional carrier in order to increase its immunogenicity, and an antiserum to the peptide-carrier conjugate raised. Coupling of a peptide to a carrier protein and immunizations may be performed as described in Dymecki et al., 1992, J. Biol. Chem., 267:4815. The serum can be titered against protein antigen by ELISA (below) or alternatively by dot or spot blotting (Boersma & Van Leeuwen, 1994, J. Neurosci. Methods, 51:317). A useful serum will react strongly with the appropriate peptides by ELISA, for example, following the procedures of Green et al., 1982, Cell, 28:477.

Techniques for preparing monoclonal antibodies are well known, and monoclonal antibodies may be prepared using a candidate antigen whose level is to be measured or which is to be either inactivated or affinity-purified, preferably bound to a carrier, as described by Arnheiter et al., 1981, Nature, 294:278.

Monoclonal antibodies are typically obtained from hybridoma tissue cultures or from ascites fluid obtained from animals into which the hybridoma tissue was introduced. Monoclonal antibody-producing hybridomas (or polyclonal sera) can be screened for antibody binding to the target protein according to methods known in the art.

B) Use of Antibodies to Detect the GPCR

A polyclonal or monoclonal antibody or fragment thereof (in subsequent discussions, “antibody” is meant to include all such forms), prepared according to the methods described above and to the references therein included, allows the detection of the presence or absence of the protein encoded by a gene comprising the polynucleotide comprising a sequence of the invention in cells, tissues, or other samples derived from them. Such an antibody preparation will be used as a diagnostic marker to detect the presence or absence of disease associated with the presence (over-, or underabundance relative to non-diseased tissue) or absence of the polypeptide encoded by the novel polynucleotide comprising the sequence disclosed.

Immunological tests rely on the use of either monoclonal or polyclonal antibodies and include enzyme-linked immunoassays (ELISA), immunoblotting and immunoprecipitation (see Voller, 1978, Diagnostic Horizons, 2:1, Microbiological Associates Quarterly Publication, Walkersville, M D; Voller et al., 1978, J. Clin. Pathol., 31:507; U.S. Reissue Pat. No. 31,006; UK Patent No. 2,019,408; Butler, 1981, Methods Enzymol., 73:482; Maggio, E. (ed.), 1980, Enzyme Immunoassay, (CRC Press, Boca Raton, Fla.) or Radioimmunoassays (RIA) (Weintraub, B., Principles of Radioimmunoassays, Seventh Training Course on Radioligand Assay Techniques, The Endocrine Society, March 1986, pp. 1-5, 46-49 and 68-78).

In addition to the methods mentioned above, organized tissues can be examined for the presence or absence of a protein produced by a polynucleotide according to the present invention using immunohistochemistry techniques. Broadly defined, immunohistochemistry is the term for the detection of specific antigens in tissue preparations. The method basically involves the steps of: 1) preparing fixed sections of the tissue of interest, immobilized on microscope slides; 2) incubation of the tissue sections with an antibody preparation specific for the antigen of interest; 3) removal of non-bound antibodies; and 4) detection of antibody-antigen complexes on the tissue sections.

Protocols for immunohistochemistry vary widely, as antigens and their recognition by particular antibody preparations differ dramatically, as do the tissue contexts of the antigen. Thus, different tissues require different methods of processing. For example, tissues may be fixed in paraformaldehyde or another fixative and embedded in paraffin wax or simply frozen prior to sectioning. In addition, the treatment of sectioned tissue will vary according to the antigen and antibody involved and according to the detection method used. Detection typically involves reaction of the bound antibody with a secondary antibody specific for a constant region domain of the antibody which reacts with the experimental antigen target (the so called “primary antibody”), but can alternatively be accomplished by labeling the primary antibody directly, such as with radiolabel, or with a fluorescence or enzyme tag (methods of antibody labeling are described in Harlow & Lane, 1989, Antibodies, Cold Spring Harbor Laboratory, Cold Spring Harbor, N.Y.). When the secondary antibody approach is used, the secondary antibody is typically conjugated to a detection moiety such as a radionuclide, an enzyme (e.g., horseradish peroxidase) or fluorescent tag. Secondary antibodies bearing moieties for detection by an array of different methods, and suitable for the detection of a wide variety of primary antibody types are commercially available. Detailed methods for sample preparation and performance of immunohistochemical analyses are described in Ausubel et al., 1992, supra, pp. 14-22 to 14-29, and in Humason, G. L., 1979, Animal Tissue Techniques, 4th ed. (W. H. Freeman & Co., San Francisco, Calif.). Decisions regarding the use of tissue processing methods and subsequent steps, such as detection, and other considerations, such as appropriate positive and negative controls, depend upon which tissue and antigen are under investigation, but may be made with a limited amount of experimentation by one skilled in the art.

It is contemplated that immunohistochemical methods will allow detection of the polypeptide encoded by the gene sequence comprising the polynucleotide of the invention in the following tissues: muscle, including but not limited to skeletal, cardiac, and smooth muscle; cells and tissues of the circulatory system, including but not limited to those of veins and arteries; cells of the skin; bone and bone forming cells; neuronal cells and tissues of the central nervous system, including but not limited to those of the brain and spine; liver cells, including but not limited to parenchymal and non-parenchymal hepatic cells; cells and tissues of the alimentary tract, including but not limited to esophagus, stomach, large and small intestines and rectum; tissues and cells of the reproductive systems, including but not limited to those of the ovary and testis, uterus, cervix, breast and prostate; cells and tissues of the genitourinary tract, including but not limited to kidney and bladder; cells and tissues of the endocrine system, including but not limited to the adrenal glands, hypothalamus, pituitary gland, and pancreas; immune system components, including but not limited to cells of bone marrow, lymphoid and myeloid lineages, B cells, T cells, NK cells, macrophages, and cells of the spleen; cells and tissues of the pulmonary system and lung; cells of the eye and cells of the auditory system.

It is contemplated that immunohistochemistry, performed as described above, may be used to correlate the presence or absence of a polypeptide encoded by a gene sequence comprising the polynucleotide of the invention with the presence of a disease that is associated with the polypeptide. In order for the antibodies of the invention to be used in such a way, it is necessary to perform immunohistochemical analysis on non-diseased tissues with the same antibody to establish the baseline levels of the polypeptide detected with the antibody. Thus the use of the antibody specific for the protein product of the invention as a diagnostic indicator may comprise the steps of: 1) performing immunohistochemical analysis on cells, tissues, or other samples derived from an individual suffering from, or thought to be suffering from a disease potentially related to the expression of the gene of this invention; and 2) comparing the immunohistochemical signal obtained with that observed in samples derived from non-diseased sources (preferably, but not necessarily derived from non-diseased tissue of the same patient). Presumably, the inappropriate presence (over- or underabundance) or absence of the protein product of the invention in samples derived from diseased tissues, relative to the level of such product in non-diseased tissues indicates that the product of the sequence is involved in or is diagnostic of a disease, or the propensity for a disease.

Nucleic Acid Detection

One embodiment of the present invention is the use of the polynucleotide sequence encoding the GPCR (SEQ ID NO: 2) of the present invention, or a sequence complementary thereto as a probe or probes which allow the detection of DNA or RNA (“target sequence”) corresponding or complementary to that of the disclosed sequence in cells, tissues, or samples derived therefrom. This embodiment will be referred to as a “probe for in situ hybridization analysis.” Thus, the sequence disclosed (SEQ ID NO: 2) allows the determination of the presence or absence of the DNA or RNA sequence disclosed in a given cell, tissue, or other sample preparation. In addition, such use allows determination of the cell or tissue-specific expression pattern of the newly identified gene encoded by the disclosed polynucleotide, as well as the levels of expression of its transcript. In turn, knowledge of the patterns and levels of expression of the gene in normal cells or tissues allows comparison with the levels seen in various disease states.

The probe may be DNA, RNA, or modified forms thereof as discussed above, and may be designed to hybridize with the sense or antisense strands of the target sequence, or both.

As embodied as a probe for in situ hybridization analysis, the probe may be of any suitable length and base composition, spanning the whole of or any number of portions of the disclosed sequence or its corresponding genomic sequence. As used herein, “suitable length and base composition” refers to the selection of probe length and base composition such that the probe will hybridize in a specific manner with the target sequence under stringent conditions. As used herein, “stringent conditions” means hybridization will occur only if there is at least 95%, preferably at least 97%, and optimally 100% identity or complementarity between the probe and the sequences it binds. Specific solution compositions and methods for hybridization under stringent conditions are described herein below.

One method of in situ hybridization involves the use of oligonucleotide probes complementary to the desired target nucleic acid sequence. Considerations involved in designing oligonucleotide probes for in situ hybridization analysis are similar to those involved in designing an oligonucleotide probe to screen a library, as discussed above. Oligonucleotides for use in in situ hybridization will generally be between 8 and 100 bases in length, preferably between 8 and 40 bases in length, and optimally between 15 and 25 bases in length.

Oligonucleotides used as probes for in situ hybridization may be naturally occurring double stranded or single stranded DNA or RNA. Alternatively, oligonucleotides may be chemically synthesized as described above or obtained from any of a number of commercial suppliers of custom oligonucleotides. It should be understood that oligonucleotides used for in situ hybridization probes may contain modifications as described above for polynucleotides.

For purposes of hybrid detection, probes are radioactively labeled by methods well known in the art. Particularly useful is ³⁵S labeling, which combines a high energy signal with high resolution. Alternatively, a hybrid is detected via non-isotopic methods. Non-isotopically labeled probes are produced by the addition of biotin or digoxigenin, fluorescent groups, chemiluminescent groups (e.g., dioxetanes, particularly triggered dioxetanes), enzymes or antibodies. Typically, non-isotopic probes are detected by fluorescence or enzymatic methods. Detection of a radiolabeled probe-target nucleic acid complex is accomplished by separating the complex from free probe and measuring the level of complex by autoradiography. If the probe is covalently linked to an enzyme, the enzyme-probe-conjugate-target nucleic acid complex will be isolated away from the free probe enzyme conjugate and a substrate will be added for enzyme detection. Enzymatic activity will be observed as a change in color development or luminescent output resulting in a 10³-10⁶-fold increase in sensitivity. An example of the preparation and use of nucleic acid probe-enzyme conjugates as hybridization probes (wherein the enzyme is alkaline phosphatase), is described in Jablonski et al., 1986, Nuc. Acids Res., 14:6115.

Two-step label amplification methodologies are known in the art. These assays are based on the principle that a small ligand (such as digoxigenin, biotin, or the like) is attached to a nucleic acid probe capable of specifically binding to a desired target sequence.

According to the method of two-step label amplification, the small ligand attached to the nucleic acid probe will be specifically recognized by an antibody-enzyme conjugate. For example, digoxigenin will be attached to the nucleic acid probe and hybridization will be detected by an antibody-alkaline phosphatase conjugate wherein the alkaline phosphatase reacts with a chemiluminescent substrate. For methods of preparing nucleic acid probe-small ligand conjugates, see Martin et al., 1990, BioTechniques, 9:762. Alternatively, the small ligand will be recognized by a second ligand-enzyme conjugate that is capable of specifically complexing to the first ligand. A well known example of this manner of small ligand interaction is the biotin-avidin interaction. Methods for labeling nucleic acid probes and their use in biotin-avidin based assays are described in Rigby et al., 1977, J. Mol. Biol., 113:237 and Nguyen et al., 1992, BioTechniques, 13:116).

Variations of the basic hybrid detection protocol are known in the art, and include modifications that facilitate separation of the hybrids to be detected from extraneous materials and/or that employ the signal from the labeled moiety. A number of these modifications are reviewed in: Matthews & Kricka, 1988, Anal. Biochem., 169:1; Landegren et al., 1988, Science, 242:229; Mittlin, 1989, Clinical Chem. 35:1819; U.S. Pat. No. 4,868,105; and in EPO Publication No. 225,807.

As an alternative to oligonucleotides, probes for in situ hybridization analysis may comprise fragments of single or double-stranded DNA or RNA comprising sequence which allows specific hybridization with a desired target sequence. As used in this context, a “fragment” refers to any polynucleotide greater in length than an oligonucleotide, up to approximately 6 kb, but preferably between approximately 100 bases and 1 kb in length. Such fragments to be used as probes can be chemically synthesized, but are preferably generated enzymatically. It should be understood that any or all of the modifications discussed above for polynucleotides can be incorporated into such fragments.

DNA probe fragments are generated enzymatically in a number of ways. For example, fragments are generated by digestion of naturally occurring DNA or of cloned recombinant DNA bearing the desired polynucleotide sequence with restriction endonucleases according to methods well known in the art. Such restriction digest-generated sequence fragments may contain, in addition to the sequences corresponding to (i.e., complementary to) the desired target sequence, sequences derived from the surrounding genomic sequence or from the recombinant DNA vector from which it was digested. Such restriction digest-generated sequence fragments are utilized as probes following labeling by means known in the art, such as radioactive labeling or non-isotopic labeling methods as discussed above for oligonucleotide probes. Further, there may be one or many label moieties incorporated per probe molecule, depending upon the method utilized to incorporate such label. For example, DNA or RNA polynucleotides may be labeled with a single labeling moiety per molecule, as in 5′ end labeling with α-³²P-ATP and T4 polynucleotide kinase, or with multiple ³²P-labeled bases, as in random-primed labeling with α-³²P-labeled deoxynucleotides and the Klenow fragment of E. coli DNA Pol I.

Another means of generating longer polynucleotide fragments for use as probes in in situ hybridization involves the use of PCR techniques. Probe sequences generated by PCR methodology are isotopically or non-isotopically labeled according to methods known in the art, as discussed above. Alternatively, PCR-generated probes are labeled during the PCR process by incorporation of one or more isotopically or non-isotopically labeled nucleoside triphosphates (or analogs) added to the reaction mixture.

It should be noted that the efficiency of in situ hybridization can be enhanced when using probes derived from longer DNA or RNA fragments by partial hydrolysis of the labeled probe preparation, usually by alkali treatment. In this context, “partial hydrolysis” is meant to be hydrolysis which results in the majority of fragments generated being shorter than the length prior to hydrolysis, but greater than or equal to a length that allows specific hybridization under a given set of hybridization conditions. Preferably, the hydrolyzed probe is 8 to 500 bases long, and more preferably 20 to 200 nucleotides in length. Thus, probes derived from longer DNA or RNA fragments may in practice comprise many DNA or RNA fragments generated from them.

Another alternative means of generating probes to detect the presence of polynucleotide bearing the sequence disclosed is through in vitro transcription of a DNA template to generate the corresponding RNA. This has the advantage that it generates a single-stranded probe which will hybridize with only the sense or the antisense strand of the target nucleic acid. The use of antisense RNA probes to detect sense RNA in situ has the added advantage that RNA:RNA hybrids are generally more stable than DNA:RNA hybrids.

There are at least two ways to make the transcription template. First, the sequence to be used to generate the probe can be inserted, using vectors and techniques known in the art, into a plasmid vector adjacent to a bacteriophage promoter (usually SP6, T7, or T3). Alternatively, the bacteriophage promoter sequence may be appended to a probe template fragment by being incorporated into the 5′ end of a PCR primer, the remainder of which is complementary to one end of a sequence used as a PCR template (which contains the desired target polynucleotide sequence). PCR is then carried out using the primer with the appended promoter, an appropriate 3′ primer, and a PCR template containing the desired polynucleotide sequence to be used as probe, to generate a PCR product bearing the bacteriophage promoter at one end. It is important to note that for the initial PCR cycles (i.e., cycles 1-5), the annealing temperature chosen is based upon the calculated Tₘ of that portion of the 5′ primer which is able to hybridize with the PCR template. In subsequent cycles, the annealing temperature is adjusted (increased) to reflect the Tₘ of the full length of the 5′ primer, which hybridizes along its full length with molecules synthesized in the initial five cycles. The promoter is situated with respect to the desired polynucleotide transcription template sequence such that either a sense or an antisense RNA transcript is generated. (It is possible to generate a template with a different bacteriophage promoter at each end, allowing the synthesis of sense and antisense transcripts from the same template, if desired).

An RNA transcript useful as a probe for in situ hybridization analysis may be generated for example, by the steps of: 1) denaturing the DNA template strands; 2) adding appropriate labeled or non-labeled ribonucleoside triphosphates or analogues thereof along with the appropriate buffers and bacteriophage RNA polymerase; 3) incubating for the appropriate time at the appropriate temperature; and 4) removal of the DNA template by digestion with DNaseI. (For specifics, see Ausubel et al., 1992, supra, pp. 14-16 to 14-17). As noted for longer DNA probes, RNA probes can be partially hydrolyzed prior to use to improve the efficiency of hybridization.

The use of a disclosed nucleotide sequence as an in situ hybridization probe to detect the presence or absence of nucleic acid corresponding (i.e. complementary) to the disclosed polynucleotide sequence in a cell, tissue, or other sample preparation, involves the steps of: 1) incubation, in the appropriate buffer, of labeled probe(s) with cells, tissue sections, or other sample preparations immobilized on glass slides or other appropriate support; 2) removal of unbound probe molecules; and 3) detection of bound probe complexes. The methods of preparation (i.e., sectioning, fixation, and pre-hybridization blocking) of cell, tissue, or other samples for in situ hybridization vary widely depending upon the characteristics of a given sample type and the form of probe to be used.

The following are examples of conditions for the preparation of histological samples. However, one skilled in the art may choose conditions appropriate for a given cell, tissue, or sample type. Tissue samples intended for use in in situ detection of either RNA or protein are fixed using conventional reagents; such samples may comprise whole or squashed cells, or may instead comprise sectioned tissue. Fixatives adequate for such procedures include, but are not limited to, formalin, 4% paraformaldehyde in an isotonic buffer, formaldehyde (each of which confers a measure of RNase resistance to the nucleic acid molecules of the sample) or a multi-component fixative, such as FAAG (85% ethanol, 4% formaldehyde, 5% acetic acid, 1% EM grade glutaraldehyde). Note that for RNA detection, water used in the preparation of an aqueous component of a solution to which the tissue is exposed until it is embedded is RNAase-free, i.e., treated with 0.1% diethylpyrocarbonate (DEPC) at room temperature overnight and subsequently autoclaved for 1.5 to 2 hours. Tissue is fixed at 4 C., either on a sample roller or a rocking platform, for 12 to 48 hours in order to allow fixative to reach the center of the sample.

Prior to embedding, samples are purged of fixative and dehydrated; this is accomplished through a series of two- to ten-minute washes in increasingly high concentrations of ethanol, beginning at 60% and ending with two washes each in 95% and 100% ethanol, followed by two ten-minute washes in xylene. Samples are embedded in one of a variety of sectioning supports, e.g., paraffin, plastic polymers or a mixed paraffin/polymer medium (e.g., Paraplast® Plus Tissue Embedding Medium, supplied by Oxford Labware). For example, fixed, dehydrated tissue is transferred from the second xylene wash to paraffin or a paraffin/polymer resin in the liquid-phase at about 58 C., then replaced three to six times over a period of approximately three hours to dilute out residual xylene, followed by overnight incubation at 58 C. under a vacuum in order to optimize infiltration of the embedding medium into the tissue. The next day, following several more changes of medium at 20 minute to 1 hour intervals also at 58 C., the tissue sample is positioned in a sectioning mold, the mold is surrounded by ice water and the medium is allowed to harden. Sections of 6 m thickness are taken and affixed to ‘subbed’ slides, which are those coated with a proteinaceous substrate material, usually bovine serum albumin (BSA), to promote adhesion. Other methods of fixation and embedding are also applicable for use according to the methods of the invention; examples of these are found in Humason, G. L., 1979, Animal Tissue Techniques, 4th ed. (W. H. Freeman & Co., San Francisco), as is frozen sectioning in Serrano et al., 1989, Dev. Biol. 132:410.

In addition to variations in the fixation and sample preparation conditions, the actual procedures and conditions for the hybridization of the probe to the sample will vary according to how the tissue/cell sample was prepared.

It is contemplated that probes derived from the sequence of the present invention may be used to detect the presence of DNA or RNA complementary in sequence to the gene sequence comprising the polynucleotide of the invention in any one or more of the following cell and tissue types: muscle, including but not limited to skeletal, cardiac, and smooth muscle; cells and tissues of the circulatory system, including but not limited to those of veins and arteries; cells of the skin; bone and bone forming cells; neuronal cells and tissues of the central nervous system, including but not limited to those of the brain and spine; liver cells, including but not limited to parenchymal and non-parenchymal hepatic cells; cells and tissues of the alimentary tract, including but not limited to esophagus, stomach, large and small intestines and rectum; tissues and cells of the reproductive systems, including but not limited to those of the ovary and testis, uterus, cervix, breast and prostate; cells and tissues of the genitourinary tract, including but not limited to kidney and bladder; cells and tissues of the endocrine system, including but not limited to the adrenal glands, hypothalamus, pituitary gland, and pancreas; immune system components, including but not limited to cells of bone marrow, lymphoid and myeloid lineages, B cells, T cells, NK cells, macrophages, and cells of the spleen; cells and tissues of the pulmonary system and lung; cells of the eye and cells of the auditory system.

Alternatively, nucleic acid probes constructed as described above may be employed in a Northern Blot analysis to detect nucleic acid sequences of the present invention. Molecular methods such as Northern analysis are well known in the art (see Sambrook et al., 1989, Molecular Cloning. A Laboratory Manual. 2nd Edition, Cold Spring Harbor Laboratory Press, Cold Spring Harbor, N.Y.). Briefly, Northern analysis may be performed on total RNA obtained from one or more cells to be examined for the presence of nucleic acid encoding the GPCR of the invention using classically described techniques. For example, total RNA samples are denatured with formaldehyde/formamide and run for two hours in a 1% agarose, MOPS-acetate-EDTA gel. RNA is then transferred to nitrocellulose membrane by upward capillary action and fixed by UV cross-linkage. Membranes are pre-hybridized for at least 90 minutes and hybridized overnight at 42° C. Post hybridization washes are performed as known in the art (Ausubel, supra). The membrane is then exposed to x-ray film overnight with an intensifying screen at −80° C. Labeled membranes are then visualized after exposure to film. The signal produced on the x-ray film by the radiolabeled cDNA probes can then be quantified using any technique known in the art, such as scanning the film and quantifying the relative pixel intensity using a computer program such as NIH Image (National Institutes of Health, Bethesda, Md.).

### Other Embodiments

The foregoing examples demonstrate experiments performed and contemplated by the present inventors in making and carrying out the invention. It is believed that these examples include a disclosure of techniques which serve to both apprise the art of the practice of the invention and to demonstrate its usefulness. It will be appreciated by those of skill in the art that the techniques and embodiments disclosed herein are preferred embodiments only that in general numerous equivlaent methods and techniques may be employed to achieve the same result.

All of the references identified hereinabove, are hereby expressly incorporated herein by reference to the extent that they describe, set forth, provide a basis for or enable compositions and/or methods which may be important to the practice of one or more embodiments of the present inventions.

### EXAMPLE 1

Cloning of the Human GPCR×10

In order to identify and clone novel human GPCR sequences related to P2Y receptors, the following approche was used. Sequences of the following GPCR: GPR8, ChemR23, HM74 and GPR14 were used as queries to search for homologies in public high-throughput genomic sequence databases (NCBI).

Using the above strategies, a novel human sequence of GPCR was identified and was named GPCR×10 (SEQ ID NO 1 (amino acid) and 2 (nucleic acid)).

In order to clone the GPCR× sequence a polymerase chain reaction (PCR) on total human genomic DNA was performed. Primers were synthetized based upon the human sequences described above and were as follows:

| | | |
|---|---|---|
| GPCR×10 fw: | 5′-CAGAGAATTCGGAGACAACCATGAATGAGCC-3′ | SEQ ID NO 7 |
| GPCR×10 rv: | 5′-TACTGGATCCCCAGGAGTTCCATTGAGGGAG-3′ | SEQ ID NO 8 |

Amplification resulted in a fragment of approximately 1 kilobase containing the entire coding sequence of the human gene. This fragment obtained was subcloned into the pCDNA3 (Invitrogen) vector for DNA sequencing analysis.

### EXAMPLE 2

Northern Blot Analysis of GPCR×10:

The tissue distribution of novel receptor transcripts was investigated by Northern blotting.

a) Procedure:

We have used a probe corresponding to the entire peptidic sequence. One commercial blot of human organs (Clontech, MTN 12: 1 μg polyA+RNA/lane) and a blot containing RNA from human and dog thyroids (generous gift from V. Van Vooren (I.R.I.B.H.N.) 10 μg of polyA+/lane) were hybridized with a probe corresponding to the entire coding sequence of the novel receptor in order to characterize its tissue distribution. The RNA from human and dog thyroids were prepared with the RNeasy kit (Quiagen). The blots were prehybridized 8 hours at 37° C. in a 50% formamide, 0.3% SDS solution and hybridized for 18 hours in the same solution supplemented with 10% dextran sulphate and the [α32P] labelled probe. The final washing conditions were 0.2×SSC and 0.1% SDS at 60° C. The blots were exposed during six days and visualized using the PhosphorImager SI (Molecular Dynamics).

b) Results:

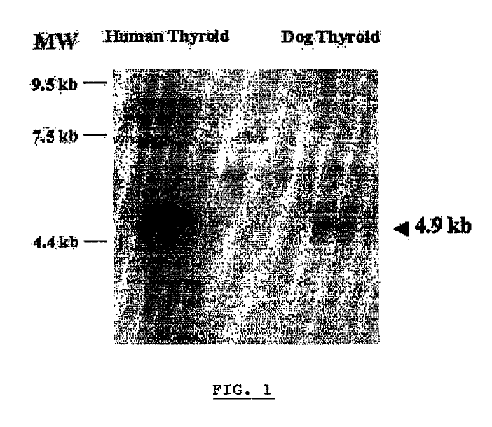

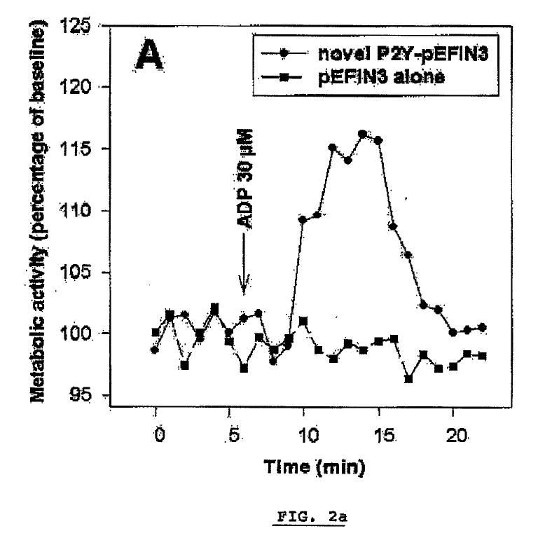

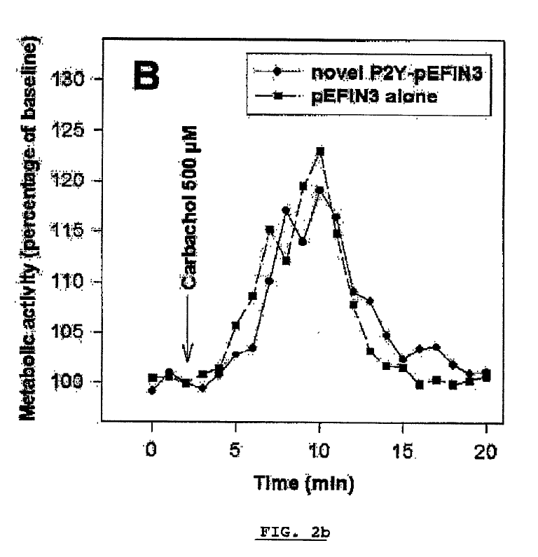

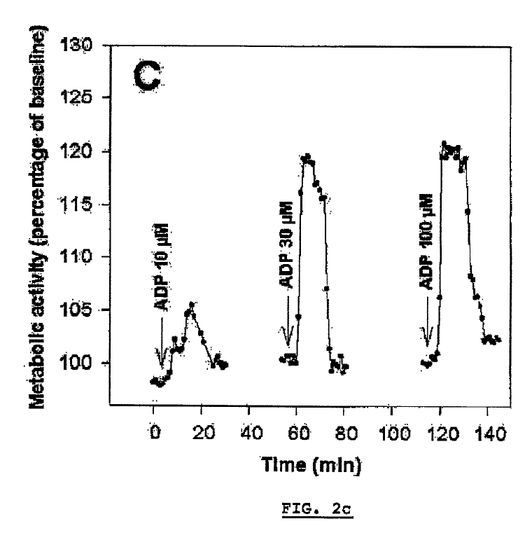

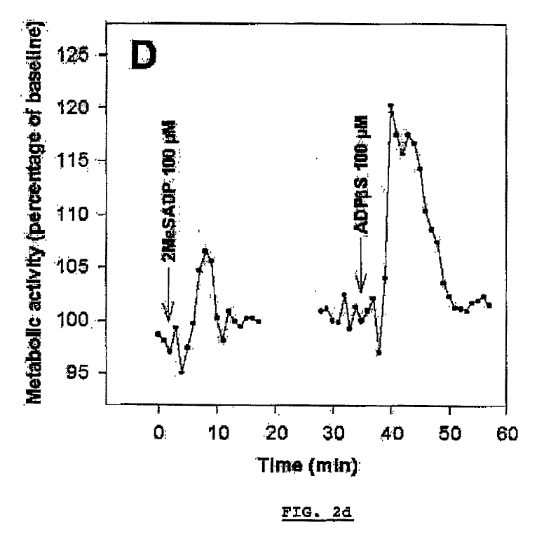

No signal was obtained on the commercial blot containing mRNA extracted from human brain, heart, skeletal muscle, colon, thymus, spleen, kidney, liver, small intestine, placenta, lung and leukocytes. However a GAPDH (glyceraldehyde phosphodehydrogenase) probe was able to reveal a specific band in all the lanes on this blot. On the contrary, a significant signal was observed on a blot containing polyA+RNA extracted from human and dog thyroids (10 μg/lane) and corresponded to a 4.9 kilobase (kb)-length messenger RNA (FIG. 1). The signal was clearly stronger in human thyroid but this was obtained with a human probe and under relatively stringent hybridization and washing conditions.

### EXAMPLE 3

Cloning of Other Human P2Y Like Receptor

In order to identify and clone novel human GPCR sequences related to P2Y receptors, the following approach was used. Sequences of the following GPCR: GPR8, ChemR23, HM74 and GPR14 were used as queries to search for homologies in public high-throughput genomic sequence databases (NCBI).

Using the above strategies, two novel human sequences of GPCR were identified:

| | |
|---|---|
| GPCRx6, | SEQ ID NO 3 |
| CPCRx13, | SEQ ID NO 5 |

In order to clone these sequences, a polymerase chain reaction (PCR) was performed on total human genomic DNA. Primers were synthetized based upon the GPCR human sequences and were as follows:

| | | |
|---|---|---|
| GPCR×6 fw: | 5′-CAGAGAATTCGTTATGCTGTCCATTTTGCTTCC-3′ | SEQ ID NO 9 |
| GPCR×10 rv: | 5′-TACTTCTAGACCCACCAGCACTCATCTGTGTAC-3′ | SEQ ID NO 10 |
| GPCR×13 fw: | 5′-CAGAGAATTCCTGCAATTCTATTCTAGCTCCTGTG-3′ | SEQ ID NO 11 |
| GPCR×13 rv: | 5′-GCGGGATCCTATTGTCAACCAAGCTGTGACATG-3′ | SEQ ID NO 12 |

Amplification resulted in a fragments of 1.14 kilobase containing the entire coding sequence of the GPCR×10 gene. This fragment was subcloned into the pCDNA3 (Invitrogen) vector for DNA sequencing analysis.

Nucleotide and deduced amino acid sequence of human GPCR×10 (SEQ ID NO; 2 and 1 respectively)

| | | |
|---|---|---|
| 1 | M N E P L D Y L A N A S D F P | 15 |
| 1 | ATG AAT GAG CCA CTA GAC TAT TTA GCA AAT GCT TCT GAT TTC CCC | 45 |
| 16 | D Y A A A F G N C T D E N I P | 30 |
| 46 | GAT TAT GCA GCT GCT TTT GGA AAT TGC ACT GAT GAA AAC ATC CCA | 90 |
| 31 | L K M H Y L P V I Y G I I F L | 45 |
| 91 | CTC AAG ATG CAC TAC CTC CCT GTT ATT TAT GGC ATT ATC TTC CTC | 135 |
| 46 | V G F P G N A V V I S T Y I F | 60 |
| 136 | GTG GGA TTT CCA GGC AAT GCA GTA GTG ATA TCC ACT TAC ATT TTC | 180 |
| 61 | K M R P W K S S T I I N L N L | 75 |
| 181 | AAA ATG AGA CCT TGG AAG AGC AGC ACC ATC ATT ATG CTG AAC CTG | 226 |
| 76 | A C T D L L Y L T S L P F L I | 90 |
| 226 | GCC TGC ACA GAT CTG CTG TAT CTG ACC AGC CTC CCC TTC CTG ATT | 270 |
| 91 | H Y Y A S G B N W I F G D F M | 105 |
| 271 | CAC TAC TAT GCC AGT GGC GAA AAC TGG ATC TTT GGA GAT TTC ATG | 315 |
| 106 | C K F I R F S F H F N L Y S S | 120 |
| 316 | TGT AAG TTT ATC CGC TTC AGC TTC CAT TTC AAC CTG TAT AGC AGC | 360 |
| 121 | I L F L T C F S I F R Y C V I | 135 |
| 361 | ATC CTC TTC CTC ACC TGT TTC AGC ATC TTC CGC TAC TGT GTG ATC | 405 |
| 136 | I H P M S C F S I H K T R C A | 150 |
| 406 | ATT CAC CCA ATG AGC TGC TTT TCC ATT CAC AAA ACT CGA TGT GCA | 450 |
| 151 | V V A C A V V W I I S L V A V | 165 |
| 451 | GTT GTA GCC TGT GCT GTG GTG TGG ATC ATT TCA CTG GTA GCT GTC | 495 |
| 166 | I P M T F L I T S T N R T N R | 180 |
| 496 | ATT CCG ATG ACC TTC TTG ATC ACA TCA ACC AAC AGG ACC AAC AGA | 540 |
| 181 | S A C L D L T S S D E L N T I | 195 |
| 541 | TCA GCC TGT CTC GAC CTC ACC AGT TCG GAT GAA CTC AAT ACT ATT | 585 |
| 196 | K W Y N L I L T A T T F C L P | 210 |
| 586 | AAG TGG TAC AAC CTG ATT TTG ACT GCA ACT ACT TTC TGC CTC CCC | 630 |
| 211 | L V I V T L C Y T T I I H T L | 225 |
| 631 | TTG GTG ATA GTG ACA CTT TGC TAT ACC ACG ATT ATC CAC ACT CTG | 675 |
| 226 | T H G L Q T C S C L K Q K A R | 240 |
| 676 | ACC CAT GGA CTG CAA ACT GAC AGC TGC CTT AAG CAG AAA GCA CGA | 720 |
| 241 | R L T I L L L L A F Y V C F L | 255 |
| 721 | AGG CTA ACC ATT CTG CTA CTC CTT GCA TTT TAC GTA TGT TTT TTA | 765 |
| 256 | P F H I L R V I R I E S R L L | 270 |
| 766 | CCC TTC CAT ATC TTG AGG GTC ATT CGG ATC GAA TCT CGC CTG CTT | 810 |
| 271 | S I S C S I E N Q I H E A Y I | 285 |
| 811 | TCA ATC AGT TGT TCC ATT GAG AAT CAG ATC CAT GAA GCT TAC ATC | 855 |
| 286 | V S R P L A A L N T F G N L L | 300 |
| 856 | GTT TCT AGA CCA TTA GCT GCT CTG AAC ACC TTT GGT AAC CTG TTA | 900 |
| 301 | L Y V V V S D N F Q Q A V C S | 315 |
| 901 | CTA TAT GTG GTG GTC AGC GAC AAC TTT CAG CAG GCT GTC TGC TCA | 945 |
| 316 | T V R C K V S G N L E Q A K K | 330 |
| 946 | ACA GTG AGA TGC AAA GTA AGC GGG AAC CTT GAG CAA GCA AAG AAA | 990 |

| | | |
|---|---|---|
| 331 | I S Y S N N P * | 338 |
| 991 | ATT AGT TAC TCA AAC AAC CCT TGA | 1014 |

Amino acid sequence of human GPCR×10 (337 amino acids) (SEQ ID NO. 1). The seven predicted transmembrane domaines are underlined.

MNEPLDYLANASDFPDYAAAFGNCTDENIPLKMHYLPVIYGIIFLVGFPGNAVVISTYIFKMRPWK
SSTIIMLNLACTDLLYLTSLPFLIHYYASGENWIFGDFMCKFIRFSFHFNLYSSILFLTCFSIFRY
CVIIHPMSCFSIHKTRCAVVACAVVWIISLVAVIPMTFLITSTNRTNRSACLDLTSSDELNTIKWY
NLILTATTFCLPLVIVTLCYTTIIHTLTHGLQTDSCLKQKARRLTILLLLAFYVCFLPFHILRVIR
IESRLLSISCSIENQIHEAYIVSRPLAALNTFGNLLLYVVVSDNFQQAVCSTVRCKVSGNLEQAKK
ISYSNNP

At the amino acid sequence level, the human GPCR×1 is 35% identical to the mouse P2Y1. GPCR×10 is located on chromosome 13.

M L S I L L P S R G S R S G S R R G A L L L E G A S R D M E K V D M N T
S Q E Q G L C Q F S E K Y K Q V Y L S L A Y S I I F I L G L P L N G T V
L W H S W G Q T K R W S C A T T Y L V N L M V A D L L Y V L L P F L I I
T Y S L D D R W P F G E L L C K L V H F L F Y I N L Y G S I L L L T C I
S V H Q F L G V C H P L C S L P Y R T R R H A W L G T S T T W A L V V L
Q L L P T L A F S H T D Y I N G Q M I W Y D M T S Q E N F D R L F A Y G
I V L T L S G F F P S L V I L V C Y S L M V R S L I K P E E N L M R T G
N T A R A R S I R T I L L V C G L P T L C F V P F H I T R S F Y L T I C
F L L S Q D C Q L L M A A S V A Y K I W R P L V S V S S C L N P V L Y F
L S R G A K I E S G S S R N

At the amino acid sequence level, the human GPCR×6 is 39% identical to the human

Nucleotide and deduced acid sequence of human GPCR×6 SEQ ID NO: 3 and 4 respectively)

| | | |
|---|---|---|
| 1 | M L S I L L P S R G S R S G S R R G A L | 20 |
| 1 | ATGCTGTCCATTTTGCTTCCTTCCAGGGGAAGCAGAAGCGGGAGCCGTCGTGGAGCTCTG | 60 |
| 21 | L L E G A S R D M E K V D M N T S Q E Q | 40 |
| 61 | CTCCTGGAGGGAGCCTCCCGGGACATGGAGAAGGTGGACATGAATACATCACAGGAACAA | 120 |
| 41 | G L C Q F S E K Y K Q V Y L S L A Y S I | 60 |
| 121 | GGTCTCTGCCAGTTCTCAGAGAAGTACAAGCAAGTCTACCTCTCCCTGGCCTACAGTATC | 180 |
| 61 | I P I L G L P L N G T V L W H S W G Q T | 80 |
| 181 | ATCTTTATCCTAGGGCTGCCACTAAATGGCACTGTCTTGTGGCACTCCTGGGGCCAAACC | 240 |
| 81 | K R W S C A T T Y L V N L M V A D L L Y | 100 |
| 241 | AAGCGCTGGAGCTGTGCCACCACCTATCTGGTGAACCTGATGGTGGCCGACCTGCTTTAT | 300 |
| 101 | V L L P F L I I T Y S L D D R W P F G E | 120 |
| 301 | GTGCTATTGCCCTTCCTCATCATCACCTACTCACTAGATGACAGGTGGCCCTTCGGGGAG | 360 |
| 121 | L L C K L V H F L F Y I N L Y G S I L L | 140 |
| 361 | CTGCTCTGCAAGCTGGTGCACTTCCTGTTCTATATCAACCTTTACGGCAGCATCCTGCTG | 420 |
| 141 | L T C I S V H Q F L G V C H P L C S L P | 160 |
| 421 | CTGACCTGCATCTCTGTGCACCAGTTCCTAGGTGTGTGCCACCCACTGTGTTCGCTGCCC | 480 |
| 161 | Y R T R R H A W L G T S T T W A L V V L | 180 |
| 481 | TACCGGACCCGCAGGCATGCCTGGCTGGGCACCAGCACCACCTGGGCCCTGGTGGTCCTC | 540 |
| 181 | Q L L P T L A F S H T D Y I N G Q M I W | 200 |
| 541 | CAGCTGCTGCCCACACTGGCCTTCTCCCACACGGACTACATCAATGGCCAGATGATCTGG | 600 |
| 201 | Y D M T S Q E N F D R L F A Y G I V L T | 220 |
| 601 | TATGACATGACCAGCCAAGAGAATTTTGATCGGCTTTTTGCCTACGGCATAGTTCTGACA | 660 |
| 221 | L S G F F P S L V I L V C Y S L M V R S | 240 |
| 661 | TTGTCTGGCTTTTTTCCCTCCTTGGTCATTTTGGTGTGCTATTCACTGATGGTCAGGAGC | 720 |
| 241 | L I X P E E N L M R T G N T A R A R S I | 260 |
| 721 | CTGATCAAGCCAGAGGAGAACCTCATGAGGACAGGCAACACAGCCCGAGCCAGGTCCATC | 780 |
| 261 | R T I L L V C G L F T L C F V P F H I T | 280 |
| 781 | CGGACCATCCTACTGGTGTGTGGCCTCTTCACCCTCTGTTTTGTGCCCTTCCATATCACT | 840 |
| 281 | R S F Y L T I C F L L S Q D C Q L L M A | 300 |
| 841 | CGCTCCTTCTACCTCACCATCTGCTTTCTGCTTTCTCAGGACTGCCAGCTCTTGATGGCA | 900 |
| 301 | A S V A Y K I W R P L V S V S S C L N P | 320 |
| 901 | GCCAGTGTGGCCTACAAGATATGGAGGCCTCTGGTGAGTGTGAGCAGCTGCCTCAACCCA | 960 |
| 321 | V L Y F L S R G A K I E S G S S R N * | |
| 961 | GTCCTGTACTTTCTTTCAAGGGGGGCAAAAATAGAGTCAGGCTCCTCCAGAAACTGA | |

Amino acid sequence of human GPCR×6 (338 amino acids) (SEQ ID NO 4. The seven predicted transmembranes domains are underlined.

Nucleotide and deduced amino acid sequence of human GPCR×13 (SEQ ID NO: 5 and 6 respectively)

| | | |
|---|---|---|
| 1 | M N N N T T C I Q P S M I S S | 15 |
| 1 | ATG AAC AAC AAT ACA ACA TGT ATT CAA CCA TCT ATG ATC TCT TCC | 45 |
| 16 | M A L P I I Y I L L C I V G V | 30 |
| 46 | ATG GCT TTA CCA ATC ATT TAC ATC CTC CTT TCT ATT GTT GGT GTT | 90 |
| 31 | F G N T L S Q W I F L T K I G | 45 |
| 91 | TTT GGA AAC ACT CTC TCT CAA TGG ATA TTT TTA ACA AAA ATA GGT | 135 |
| 46 | K K T S T H I Y L S H L V T A | 60 |
| 136 | AAA AAA ACA TCA ACG CAC ATC TAC CTG TCA CAC CTT GTG ACT GCA | 180 |
| 61 | N L L V C S A M P F M S I Y F | 75 |
| 181 | AAC TTA CTT GTG TGC AGT GCC ATG CCT TTC ATG AGT ATC TAT TTC | 225 |
| 76 | L K G F Q W E Y Q S A Q C R V | 90 |
| 226 | CTG AAA GGT TTC CAA TGG GAA TAT CAA TCT GCT CAA TGC AGA GTG | 270 |
| 91 | V N F L G T L S M H A S M F V | 105 |
| 271 | GTC AAT TTT CTG GGA ACT CTA TCC ATG CAT GCA AGT ATG TTT GTC | 315 |
| 106 | S L L I L S W I A I S R Y A T | 120 |
| 316 | AGT CTC TTA ATT TTA AGT TGG ATT GCC ATA AGC CGC TAT GCT ACC | 360 |
| 121 | L M Q K D S S Q E T T S C Y E | 135 |
| 361 | TTA ATG CAA AAG GAT TCC TCG CAA GAG ACT ACT TCA TGC TAT GAG | 405 |
| 136 | K I F Y G H L L K K F R Q P N | 150 |
| 406 | AAA ATA TTT TAT GGC CAT TTA CTG AAA AAA TTT CGC CAG CCC AAC | 450 |
| 151 | F A R K L C I Y I W G V V L G | 165 |
| 451 | TTT GCT AGA AAA CTA TGC ATT TAC ATA TGG GGA GTT GTA CTG GGC | 495 |
| 166 | I I I P V T V Y Y S V I E A T | 180 |
| 496 | ATA ATC ATT CCA GTT ACC GTA TAC TAC TCA GTC ATA GAG GCT ACA | 540 |
| 181 | E G E E S L C Y N R Q M E L G | 195 |
| 541 | GAA GGA GAA GAG AGC CTA TGC TAC AAT CGG CAG ATG GAA CTA GGA | 585 |
| 196 | A M I S Q I A G L I G T T F I | 210 |
| 586 | GCC ATG ATC TCT CAG ATT GCA GGT CTC ATT GGA ACC ACA TTT ATT | 630 |
| 211 | G F S F L V V L T S Y Y S F V | 225 |
| 631 | GGA TTT TCC TTT TTA GTA GTA CTA ACA TCA TAC TAC TCT TTT GTA | 675 |
| 226 | S H L R K I R T C T S I M E K | 240 |
| 676 | AGC CAT CTG AGA AAA ATA AGA ACC TGT ACG TCC ATT ATG GAG AAA | 720 |
| 241 | D L T Y S S V K R H L L V I Q | 255 |
| 721 | GAT TTG ACT TAC AGT TCT GTG AAA AGA CAT CTT TTG GTC ATC CAG | 765 |
| 256 | I L L I V C F L P Y S I F K P | 270 |
| 766 | ATT CTA CTA ATA GTT TGC TTC CTT CCT TAT AGT ATT TTT AAA CCC | 810 |
| 271 | I F Y V L H Q R D N C Q Q L N | 285 |
| 811 | ATT TTT TAT GTT CTA CAC CAA AGA GAT AAC TGT CAG CAA TTG AAT | 855 |
| 286 | Y L I E T K N I D T C L A S A | 300 |
| 856 | TAT TTA ATA GAA ACA AAA AAC ATT CTC ACC TGT CTT GCT TCG GCC | 900 |
| 301 | R S S T D P I I F L L L D K T | 315 |
| 901 | AGA AGT AGC ACA GAC CCC ATT ATA TTT CTT TTA TTA GAT AAA ACA | 945 |
| 316 | F K K T L Y N L F T K S N S A | 330 |
| 946 | TTC AAG AAG ACA CTA TAT AAT CTC TTT ACA AAG TCT AAT TCA GCA | 990 |

| | | |
|---|---|---|
| 331 | H M Q S Y G * | 337 |
| 881 | CAT ATG CAA TCA TAT GGT TGA | 1011 |

Amino acid sequence of human GPCR×13 (335 amino acids) (SEQ ID NO: 6 The six predicted transmembrane domaines are underlined.

MNNNTTCIQPSMISSMALPIIYILLCIVGVFGNTLSQWIFLTKIGKKTSTHIYLSHLVTANLLVCSAMPFMSIYFLKGFQ
WEYQSAQCRVVNFLGTLSMHASMFVSLLILSWIAISRYATLMQKDSSQETTSCYEKIFYGHLLKKFRQPNFARKLCIYIW
GVVLGIIIPVTVYYSVIRATRGEESLCYNRQMELGAMISQIAGLIGTTFIGFSFLVVLTSYYSFVSHLRKIRTCTSIMEK
DLTYSSVKRHLLVIQILLIVCFLPYSIFKPIFYVLHQRDNCQQLNYLIETKNILTCLASARSSTDPIIFLLLDKTRKKTL
YNLFTKSNSAHMQSYG

At the amino acid sequence level, the human GPCR×13 is 26% identical to the human GPR17.

1. A G-protein coupled receptor having an amino acid sequence which presents more than 75% sequence identity with the sequence SEQ ID NO. 1.

2. The G-protein coupled receptor according to claim 1, having an amino acid sequence which presents more than 80% sequence identity with the sequence SEQ ID NO. 1.

3. The G-protein coupled receptor according to claim 1, having an amino acid sequence which presents more than 85% sequence identity with the sequence SEQ ID NO. 1.

4. The G-protein coupled receptor according to claim 1, having an amino acid sequence which presents more than 90% sequence identity with the sequence SEQ ID NO. 1.

5. The G-protein coupled receptor according to claim 1, having an amino acid sequence which presents more than 95% sequence identity with the sequence SEQ ID NO. 1.

6. The G-protein coupled receptor having the amino acid sequence SEQ ID NO. 1.

7. A polynucleotide encoding any of the amino acid sequences of the G-protein coupled receptor according to any of the preceding claims 1 to 6.

8. A vector comprising the polynucleotide according to the claim 7.

9. A cell transformed by the vector according to the claim 8.

10. A non-human mammal comprising a partial or total deletion of the polynucleotide according to the claim 7 encoding the receptor according to claim 1, preferably an non-human mammal comprising an homologous recombination “knock-out” of said polynucleotide or a transgenic non-human mammal overexpressing above natural level said polynucleotide.

11. A method for the screening of a compound which is an agonist, reverse agonist, or antagonist of the receptor according to claim 1, said method comprising:
(a) contacting a cell or cell extract from the cell transfected with a vector according to the claim 8 and;
(b) detecting the presence of any such compound by means of a bioassay in the presence of the other known compound working as an agonist, reverse agonist, or antagonist to the receptor and thereby recovering and determining whether said unknown compound or molecule(s) is able to work as an agonist, reverse agonist, or antagonist of the compound to its receptor.

12. The method of claim 11, further comprising isolating a membrane fraction from the cell extract or the complete cell with a compound binding to said receptor under conditions permitting binding of said compound or molecules present in said natural extract to said receptor.

13. The method of claim 12 wherein said binding comprises activation of a functional response.

14. The method of claim 11, wherein said bioassay comprises a modification in the production of a second messenger or an increase in the receptor activity.

15. A diagnostic kit comprising a protein recognition molecule for the detection of the receptor of claim 1, 2, 3, 4, 5, or 6 and packaging materials therefor.

16. The diagnostic kit of claim 15, wherein said receptor has the amino acid sequence set forth in SEQ ID NO: 1.

17. A diagnostic kit comprising a nucleic acid probe for the detection of a polynucleotide sequence encoding the receptor of claim 1, 2, 3, 4, 5, or 6, and packaging materials therefor.

18. The diagnostic kit of claim 17, wherein said polynucleotide sequence is the polynucleotide sequence of SEQ ID NO: 2.
